(19) United States
(12) Patent Application Publication
(10) Pub. No.: US 2024/0325390 A1
(43) Pub. Date: Oct. 3, 2024

(54) IMATINIB FORMULATIONS, MANUFACTURE, AND USES THEREOF

(71) Applicant: Aerovate Therapeutics, Inc., Boston, MA (US)

(72) Inventors: Ben Dake, Boston, MA (US); Ralph Niven, Portola Valley, CA (US)

(73) Assignee: Aerovate Therapeutics, Inc., Boston, MA (US)

(21) Appl. No.: 18/034,558

(22) PCT Filed: Nov. 23, 2021
(86) PCT No.: PCT/US21/60526

§ 371 Date: Apr. 28, 2023

Related U.S. Application Data
(60) Provisional application No. 63/117,258, filed on Nov. 23, 2020.
(60) Provisional application No. 63/150,731, filed on Feb. 18, 2021.

(51) Int. Cl. A61K 31/506; A61K 9/00; A61K 47/26

(52) U.S. Cl. CPC A61K 31/506; A61K 9/0075; A61K 47/26

(57) ABSTRACT

The invention relates to inhalable imatinib formulations, manufacture, and uses thereof.

### CROSS-REFERENCE TO RELATED APPLICATIONS

This application claims the benefit of U.S. Provisional Application Ser. No. 63/117,258, filed Nov. 23, 2020, and U.S. Provisional Application Ser. No. 63/150,731, filed Feb. 18, 2021, the content of each of which is incorporate herein by reference in its entirety.

### FIELD OF THE INVENTION

The invention generally relates to inhalable formulations of imatinib and their manufacture and uses.

### BACKGROUND

Pulmonary arterial hypertension (PAH) includes a group of rare, chronic cardiopulmonary diseases with various etiologies that share the common pathologic features of inappropriate cell growth resulting in the increased resistance to blood flow through the pulmonary vasculature. WHO Group 1 PAH is a rare disease with various etiologies that is caused by the abnormal accumulation of various cell types (smooth muscle, endothelial pericytes, fibroblasts, and myofibroblasts) in the pulmonary arteries. The disease is progressive and, if left untreated, results in an increase in right ventricular afterload impairing right ventricular function and leading to heart failure and death. With the introduction of pulmonary vasodilator therapy for the treatment of patients with PAH, median survival has improved from 2.8 years without treatment to 7-9 years in the current era of PAH-specific therapies. However, there is no cure for the disease, and there are no therapies available that directly target the proliferative nature of the pulmonary vasculopathy.

Imatinib, a tyrosine kinase inhibitor approved for the treatment of patients with chronic myeloid leukemia, has demonstrated therapeutically significant improvements on meaningful measures in clinical trials of PAH patients. Clinical efficacy of oral imatinib mesylate in the treatment of PAH was observed in a subset of PAH patients in a Phase 2 trial and confirmed in functional class II-IV patients in the Phase 3 IMPRES trial, in which the primary end point, 6-minute walk distance (6MWD), as well as secondary endpoints measuring pulmonary vascular resistance (PVR), mean pulmonary artery pressure (mPAP), cardiac output (CO), and N-terminal (NT)-pro hormone brain natriuretic peptide (NT-proBNP) all showed statistically significant and therapeutically relevant improvements on top of the maximal standard of care.

Unfortunately, an unacceptable amount of severe adverse events including subdural hematoma blunted enthusiasm for the drug. Frost, et al., 2015, Long-term safety and efficacy of imatinib in pulmonary arterial hypertension, J Heart Lung Transplant, 34 (11):1366-75, incorporated herein by reference.

### SUMMARY

Compositions and methods of the invention address problems with previous imatinib-based PAH treatments through the use of inhalable formulations. Particularly, the invention recognizes that PAH and other conditions can be effectively treated via inhalable formulations with significantly lower doses than when administered orally or intravenously. While published studies of oral imatinib mesylate for the treatment of PAH relied on administration of between 200-400 mg/day of imatinib mesylate, inhalable formulations of the invention can be provided in doses as small as 1 mg. In various embodiments, doses may be provided once a day or twice a day in amounts such as 1 mg, 3 mg, 10 mg, 30 mg, or 90 mg, all substantially lower than the 200-400 mg in the oral studies mentioned above. In preferred embodiments, doses are provided twice a day in amounts of 10 mg, 35 mg, or 70 mg. Accordingly, formulations and methods of the invention result in lower systemic concentrations than those oral treatments and reduce the risk of adverse events experienced in those studies. Lower effective doses may be facilitated through the direct delivery of imatinib formulations to target lung tissue via inhalation, the use imatinib free base, simplified compositions including only imatinib and a carrier such as lactose, and/or efficient lung penetration through the use of micronized dry powder imatinib.

The compositions and methods described herein represent a significant advancement in the treatment of PAH and other conditions of the pulmonary cardiovascular system, offering therapeutic results without the unacceptable risk of adverse events associated with prior oral formulations.

In various embodiments, imatinib formulations may comprise micronized dry powder compositions of imatinib free base blended with a lactose carrier. These compositions can be provided in doses ranging from 1 mg to 90 mg to treat subjects with pulmonary cardiovascular system conditions such as pulmonary arterial hypertension (PAH). Doses may be administered 1, 2, 3, 4, or more times a day. For example, a 90 mg dose, administered twice daily would result in a 180 mg daily dose.

Aspects of the invention may include an inhalable formulation comprising at least 1 mg of imatinib. Imatinib may be present in the formulation in an amount such as 1 mg, 3, mg, 10 mg, 35 mg, 70 mg, or 90 mg. The formulation may comprise a carrier. The carrier may be lactose. The formulation may be a dry powder. In certain aspects, a combination product may be provided comprising a dry powder inhaler and a dry powder formulation comprising at least 1 mg of imatinib. The formulation may be packaged in a capsule sized and configured to be inserted into the dry powder inhaler.

Certain aspects of the invention include methods of treating a condition of the pulmonary cardiovascular system by providing to a human subject having such a condition an inhalable formulation comprising at least 1 mg of imatinib. The formulation may be provided one daily or twice daily. In certain embodiments, a dry powder inhaler loaded with the imatinib formulation may be provided.

In certain embodiments, the imatinib dry powder may be micronized and the micronized imatinib dry powder may consist of particles comprising a mass median aerodynamic diameter in the range of about 0.5 to about 5 μm.

In certain embodiments, the invention provides a formulation of inhalable imatinib with a higher ratio of API (active pharmaceutical ingredient) than found in conventional formulations. In certain embodiments, formulations comprising 50% or more imatinib by mass are provided. Compositions and methods of the invention recognize that large volumes may be difficult or dangerous for patients to inhale and that, therefore, minimizing the amount of non-API components in the formulation can improve patient comfort, safety, and compliance by reducing the overall amount of compound that is inhaled while still providing a therapeutically effective API concentration in target tissue.

Inhalable compositions of the invention offer greater lung exposure than equivalent doses of imatinib or imatinib mesylate administered through conventional oral routes or by IV. While a relatively high oral dose of imatinib or imatinib mesylate would be required to achieve the same target lung exposure as achieved by inhalation of the inventive formulations, significantly lower dosages can be delivered using the inventive compositions and methods.

In certain embodiments inhalable imatinib compounds may be micronized through wet or dry milling (e.g., jet milling) to achieve the desired particle size for dry powder formulations for inhalation. Imatinib may be micronized to particle sizes of about 0.5 μm to about 5 μm mass median aerodynamic diameter (MMAD) for desired deep lung penetration. Inhaled products can be limited in terms of mass of powder that can be administered and certain imatinib salts will contribute significantly to the molecular weight of the inhaled compound. Accordingly, in certain embodiments, the imatinib free base may be preferred for efficient delivery of the active moiety to lung tissue. In preferred embodiments, the micronized dry powder imatinib free base is blended with a lactose carrier. API: carrier ratios may be greater than 50:50, 75:25, or 90:10. In certain embodiments, the 100% imatinib may be administered with no carrier.

Because the inhalable formulations described herein can modulate the uptake of imatinib in the target tissue of the lungs or microvasculature, formulations of the invention can be used to treat various conditions of the pulmonary cardiovascular system while avoiding the adverse events associated with higher doses that are administered by other routes of administration that introduce the drug systemically prior to reaching the target tissue. For example, compounds and methods of the invention can be used to treat PAH as well as lung transplant rejection, pulmonary veno-occlusive disease (PVOD) and pulmonary hypertension secondary to other diseases like heart failure with preserved ejection fraction (HFpEF) or schistosomiasis. Dose ranges can include between about 1 mg to about 100 mg per dose for inhalation on a daily schedule. Once or twice daily doses may comprise 1 mg, 3, mg, 10 mg, 30 mg, 35 mg, 70 mg, or 90 mg in preferred embodiments. In various embodiments, between 1 mg and 300 mg may be provided in a dose of inhalable imatinib. About 0.01 mg to about 240 mg of the active imatinib compound may then be deposited within the lungs after inhalation. Because compositions of the invention can have relatively high concentrations of API (e.g., 50% or greater), the above doses can be achieved with less overall mass of inhalable powder compared to conventional formulations having 1%-3% w/w API.

In certain embodiments, formulations of the invention can include processing and administration of imatinib in free base form. Free base imatinib formulations of the invention can retain crystallinity after micronization and are less hygroscopic than certain imatinib salts. Accordingly, compounds and methods of the invention include inhalable formulations of free base imatinib.

In some embodiments, inhalable dry powder imatinib may be provided along with methods of delivering such formulations through inhalation such as a dry powder inhaler. The imatinib may be present in a therapeutically effective amount to treat a condition of the pulmonary cardiovascular system, such as pulmonary arterial hypertension (PAH).

### BRIEF DESCRIPTION OF THE DRAWINGS

FIG. 1 shows a comparison of mean imatinib concentrations in plasma and the lungs over time.

FIG. 2 shows a comparison of mean free imatinib concentrations in plasma and the lungs over time.

FIG. 3 shows a comparison of plasma concentration over time for intratracheally administered imatinib suspension (IT) and dry powder imatinib (IT DP) to orally administered imatinib (PO).

FIG. 4 shows a comparison of lung concentrations over time for intratracheally administered imatinib suspension (IT) and dry powder imatinib (IT DP) to orally administered imatinib (PO).

FIG. 5 shows imatinib plasma concentration over time for the various cohorts in the SAD study (standard and semi-logarithmic scales).

FIG. 6 shows plasma concentration of the imatinib metabolite N-desmethyl imatinib over time for the various cohorts in the SAD study (standard and semi-logarithmic scales).

FIG. 7 shows imatinib plasma concentration of a 90 mg steady state dose compared to 400 mg oral single and steady state doses (estimated).

FIG. 8 shows imatinib plasma concentration of a 90 mg steady state dose at day 7 of the multiple ascending dose study compared to 400 mg oral single dose observed on day 1 and concentration for a projected second dose at 90 mg on day 7.

FIG. 9 shows N-desmethyl imatinib plasma concentration of a 90 mg steady state dose compared to 400 mg oral single and steady state doses (estimated).

FIG. 10 shows N-desmethyl imatinib plasma concentration of a 90 mg dose at day 7 of the multiple ascending dose study compared to 400 mg oral single and steady state doses (projected).

FIG. 11 shows concentration by time following multiple administration of inhaled AV-101 or simulated profile for 400 mg once daily imatinib with a semi-logarithmic scale for imatinib.

FIG. 12 shows concentration by time following multiple administration of inhaled AV-101 or simulated profile for 400 mg once daily imatinib with a semi-logarithmic scale for N-desmethyl imatinib.

### DETAILED DESCRIPTION

The invention relates to inhalable formulations of imatinib free base. Inhalable formulations may comprise at least on 1 mg of imatinib and may be used once-a-day or twice-a-day. In some embodiments, doses may be administered 3, 4, or more times a day. Individual doses may consist of 1 mg, 3 mg, 10 mg, 30 mg, or 90 mg. In certain embodiments, doses may comprise less than 100 mg, less than 150 mg, less than 200 mg, less than 250 mg, or less than 300 mg provided once, twice, three, four, or more times daily. Formulations may comprise imatinib free base, which may be micronized, and may be blended with a carrier such as lactose. The formulation may by a dry powder and may be provided in a capsule for use in a compatible dry powder inhaler. Preferred doses may comprise 10 mg, 35 mg, or 70 mg provided twice a day.

Such formulations may be used to treat conditions of the pulmonary cardiovascular system including PAH. While significantly higher oral doses of imatinib mesylate have previously showed promise in treating PAH, unacceptable levels of adverse events were associated with their use and, as such, they were not found safe to use. Unexpectedly, the inhalable formulations of imatinib described herein, in the aforementioned doses, can still provide therapeutic benefit in the treatment of PAH while avoiding the adverse events associated with oral imatinib mesylate formulations. It is believed that direct delivery to target lung tissue allows a therapeutic concentration of the drug to be achieved while avoiding the systemic concentrations associated with adverse effects such as subdural hematoma.

As discussed in Example 2 below, inhalation affords a high local concentration in the lung with a significantly lower plasma concentration, confirming a reduction in the risk of adverse events associated with high systemic concentrations caused by other administration routes. Furthermore, as shown in Example 2, dry powder inhalation as opposed to an inhaled suspension or solution can exhibit improved lung retention in the rat lung.

In various embodiments, the imatinib formulations of the invention may be pharmaceutical compositions for use in treating various conditions of the pulmonary cardiovascular system, such as PAH. For example, imatinib is a potent inhibitor of the platelet-derived growth factor receptor (PDGFR) and other signaling kinases. Accordingly, the compositions of the invention may be used to treat any disease or disorder that involves inhibition of PDGFR or other kinases sensitive to imatinib. In certain embodiments, treatment compositions and methods are as described below in Example 1.

In certain embodiments, the compositions of the invention may be used to treat PAH. For treatment of PAH or other disorders, a therapeutically effective amount of a pharmaceutical composition of imatinib according to the various embodiments described herein can be delivered, via inhalation (e.g., via dry powder inhaler or nebulizer) to deliver the desired amount of imatinib compound to the target lung tissue.

Dosages for treating PAH and other conditions of the pulmonary cardiovascular system may be in the range of between about 1 mg to about 300 mg per dose for inhalation on once, twice or three times per day schedule. About 0.01 mg to about 240 mg of the imatinib may then be deposited within the lung after inhalation. In certain embodiments about 1 mg to 40 mg of imatinib may be given in a capsule for a single dry-powder inhalation dose with about .5 mg to about 20 mg of the compound to be expected to reach the lungs. Multiple capsules may be used to administer a single dose. For example, 1 mg or 10 mg or 30 mg capsules may be used to constitute a single dose of 3 mg, 30 mg, 90, or 180 mg. In preferred embodiments, a 30 mg capsule of 100% imatinib with no carrier may be used. As noted, single doses may be administered 1, 2, 3, 4, or more times a day. In certain embodiments, a 35 mg capsule may be used such that 35 mg doses or 70 mg doses (achieved using two capsules) may be delivered twice daily.

In certain embodiments, imatinib formulations of the invention may be used to treat pulmonary hypertension as a result of schistosomiasis. See, for example, Li, et al., 2019, The ABL kinase inhibitor imatinib causes phenotypic changes and lethality in adult Schistosoma japonicum, Parasitol Res., 118(3): 881-890; Graham, et al., 2010, Schistosomiasis-associated pulmonary hypertension: pulmonary vascular disease: the global perspective, Chest, 137(6 Suppl):20S-29S, the content of each of which is incorporated herein by reference.

Imatinib pharmaceutical compositions of the invention may be used to treat lung transplant recipients to prevent organ rejection. See, Keil, et al., 2019, Synergism of imatinib, vatalanib and everolimus in the prevention of chronic lung allograft rejection after lung transplantation (LTx) in rats, Histol Histopathol, 1:18088, incorporated herein by reference.

In certain embodiments, pharmaceutical compositions described herein can be used to treat pulmonary veno-occlusive disease (PVOD). See Sato, et al., 2019, Beneficial Effects of Imatinib in a Patient with Suspected Pulmonary Veno-Occlusive Disease, Tohoku J Exp Med. 2019 Feb;247(2):69-73, incorporated herein by reference.

For treatment of any conditions of the pulmonary cardiovascular system for which imatinib may produce a therapeutic effect, compounds and methods of the invention may be used to provide greater concentration at the target lung tissue through inhalation along with consistent, predictable pharmacokinetics afforded by low polymorphism and amorphous content. The efficient localization of therapeutic compound at the target tissue allows for lower systemic exposure and avoidance of the adverse events associated with prolonged oral administration of imatinib mesylate.

The effective dosage of each agent can readily be determined by the skilled person, having regard to typical factors such as the age, weight, sex and clinical history of the patient. In general, a suitable daily dose of a compound of the invention will be that amount of the compound which is the lowest dose effective to produce the desired therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms.

The pharmaceutical compositions of the invention include a “therapeutically effective amount” of one or more of the compounds of the present invention, or functional derivatives thereof. An “effective amount” refers to an amount effective, at dosages and for periods of time necessary, to achieve the desired therapeutic result, e.g., a diminishment or prevention of effects associated with PAH. A therapeutically effective amount of a compound of the present invention or functional derivatives thereof may vary according to factors such as the disease state, age, sex, and weight of the subject, and the ability of the therapeutic compound to elicit a desired response in the subject. A therapeutically effective amount is also one in which any toxic or detrimental effects of the therapeutic agent are outweighed by the therapeutically beneficial effects.

Dosage regimens may be adjusted to provide the optimum desired response (e.g. a therapeutic or prophylactic response). For example, a single inhalable bolus may be administered, several divided doses may be administered over time or the dose may be proportionally reduced or increased as indicated by the exigency of the therapeutic situation. Actual dosage levels of the active ingredients in the pharmaceutical compositions of this invention may be varied so as to obtain an amount of the active ingredient which is effective to achieve the desired therapeutic response for a particular subject, composition, and mode of administration, without being toxic to the patient. Exemplary trials for the determination of safe dosages are described below in Example 1.

The term “dosage unit” as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the compound, and (b) the limitations inherent in the art of compounding such an active compound for the treatment of sensitivity in individuals.

In some embodiments, the therapeutically effective amount can be estimated initially either in cell culture assays or in animal models, usually rats, non-human primates, mice, rabbits, dogs, or pigs. The animal model is also used to achieve a desirable concentration range and route of administration. Such information can then be used to determine useful doses and routes for administration in other subjects. Generally, the therapeutically effective amount is sufficient to reduce PAH symptoms in a subject. In some embodiments, the therapeutically effective amount is sufficient to eliminate PAH symptoms in a subject.

Dosages for a particular patient can be determined by one of ordinary skill in the art using conventional considerations, (e.g. by means of an appropriate, conventional pharmacological protocol). A physician may, for example, prescribe a relatively low dose at first, subsequently increasing the dose until an appropriate response is obtained. The dose administered to a patient is sufficient to affect a beneficial therapeutic response in the patient over time, or, e.g., to reduce symptoms, or other appropriate activity, depending on the application. The dose is determined by the efficacy of the particular formulation, and the activity, stability, or half-life of the compounds of the invention or functional derivatives thereof, and the condition of the patient, as well as the body weight or surface area of the patient to be treated. The size of the dose is also determined by the existence, nature, and extent of any adverse side-effects that accompany the administration of a particular vector, formulation, or the like in a particular subject. Therapeutic compositions comprising one or more compounds of the invention or functional derivatives thereof are optionally tested in one or more appropriate in vitro and/or in vivo animal models of disease, such as models of PAH, to confirm efficacy, tissue metabolism, and to estimate dosages, according to methods well known in the art. In particular, dosages can be initially determined by activity, stability or other suitable measures of treatment vs. non-treatment (e.g., comparison of treated vs. untreated cells or animal models), in a relevant assay. Administration can be accomplished via single or divided doses.

Imatinib formulations preferably comprise imatinib free base and, as used throughout the application, imatinib will refer to the free base compound unless a salt thereof is recited. Imatinib as the free base has the following structure:

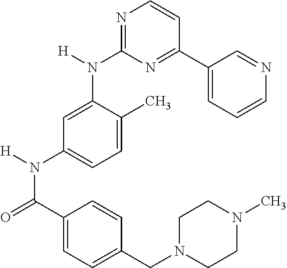 [SMILES: Cc1ccc(NC(=O)c2ccc(CN3CCN(C)CC3)cc2)cc1Nc1nccc(-c2cccnc2)n1]

In certain embodiments, imatinib formulations of the invention may include an excipient. A preferred excipient is lactose in various forms (e.g., roller dried or spray dried). Larger lactose particles can be used as a carrier for inhalation of micronized imatinib formulations. The carrier particles, with their larger size, can be used to increase aerodynamic forces on the combined imatinib/carrier in order to aid in delivery through inhalation. Solvents may be used to condition the lactose surface such that the active component can be effectively separated from the lactose as it leaves the inhaler device and within the oral cavity when being used as a carrier. In preferred embodiments, milled lactose such as Lactohale 206 available from DFE Pharma (Goch, Germany) may be used.

In various embodiments, imatinib is provided in dry powder formulations for inhalation. Dry powder can be administered via, for example, dry powder inhalers such as described in Berkenfeld, et al., 2015, Devices for Dry Powder Drug Delivery to the Lung, AAPS PharmaSciTech, 16(3):479-490, incorporated herein by reference. Dry powder compounds may be divided into single doses for single, twice daily, three times daily, or four times daily inhalation to treat disorders such as PAH or other conditions of the pulmonary cardiovascular system. The single doses may be divided into individual capsules or other formats compatible with the dry powder inhaler to be used.

Dry powder imatinib may be micronized to achieve a desired particle size. Micronized imatinib particle size can range from about 0.5 μm to about 5 μm depending on application. In preferred embodiments the size range is about 1 μm to about 3.5 μm in dry powder formulations to achieve deep lung penetration.

As noted above, the methods and compositions described herein provide greater concentrations of imatinib in target lung tissue than obtained with equivalent doses administered orally or through IV. Furthermore, those doses, comprising a high percentage of the overall formulation, are deliverable in lower volume formulations than conventional formulations of between 1% and 3% API. Reducing the powder mass that a patient must inhale can increase patient comfort and compliance, thereby improving results. Accordingly, methods and compositions of the invention allow for treatment of conditions of the pulmonary cardiovascular system (e.g., PAH) with lower doses and less inhalable drug than would be required in systemic administration, thereby lowering the risk of adverse events including subdural hematoma (See, Frost et al.). Thus, the invention provides viable treatment methods for life threatening diseases that were heretofore too risky for practical application.

In certain embodiments, compounds of the invention include formulations of imatinib. In preferred embodiments, the free base imatinib is used in a formulation (either in dry powder or suspension) for inhalation to treat a condition of the pulmonary cardiovascular system such as PAH.

When the compounds of the present invention are administered as pharmaceuticals, to humans and mammals, they can be given alone or as a pharmaceutical composition containing, for example, 0.1 to 100% of active ingredient (e.g., imatinib).

In high-API-ratio formulations, carriers or excipients may make up the remainder of the formulation in amounts of 50% or less of the overall composition. In certain embodiments, inhalable formulations may have API: carrier ratios of 50:50, 55:45, 60:40, 65:35, 70:30, 75:25, 80:20, 85:15, 90:10, or 95:5. Certain inhalable formulations may be pure API with no additional components. In various embodiments, formulations may include imatinib as the API in amounts greater than 5%, 10%, 15%, 20%, 25%, 30%, 35%, 40%, 45%., 50%, 55%, 60%, 65%, 70%, 75%, 80%, 85%, 90%, or 95%. In certain embodiments, the inhalable formulation may consist entirely of imatinib with no carrier. As used herein, API ratios refer to % w/w.

Exemplary imatinib formulations, treatment methods, and methods of production are described, for example, in U.S. patent application Ser. No. 16/874,111; Ser. No. 16/874,118; Ser. No. 16/874,122; Ser. No. 16/874,128; Ser. No. 16/874,143; Ser. No. 16/874,153; Ser. No. 16/874,168; and Ser. No. 16/874,190, the content of each of which is incorporated herein by reference.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### EXAMPLES

### Example 1: Randomized, Double-Blind, Placebo-Controlled, Single and Multiple Ascending Dose Study to Evaluate the Safety, Tolerability, and Pharmacokinetics of AV-101 (Imatinib Free Base) in Healthy Subjects

AV-101 (imatinib free base), a tyrosine kinase inhibitor administered by inhalation is studied in two phases: a single ascending dose (SAD) phase and a multiple ascending dose (MAD) phase.

### Part 1: Single Ascending Dose (SAD) Study in Healthy Volunteers.

The SAD portion included five cohorts with eight participants in each cohort (6 active: 2 placebo subjects). A single dose was administered in the morning by inhalation using a dry powder inhaler. The progression was 1 mg, 3 mg, 10 mg, 30 mg, and 90 mg. Additional cohorts may be added if the non-tolerated dose (NTD) has not been reached. The SAD portion also included one additional cohort of 8 active subjects (no placebo) who received a single oral dose of 400 mg imatinib mesylate.

Blood samples for pharmacokinetic (PK) evaluation were taken prior to and

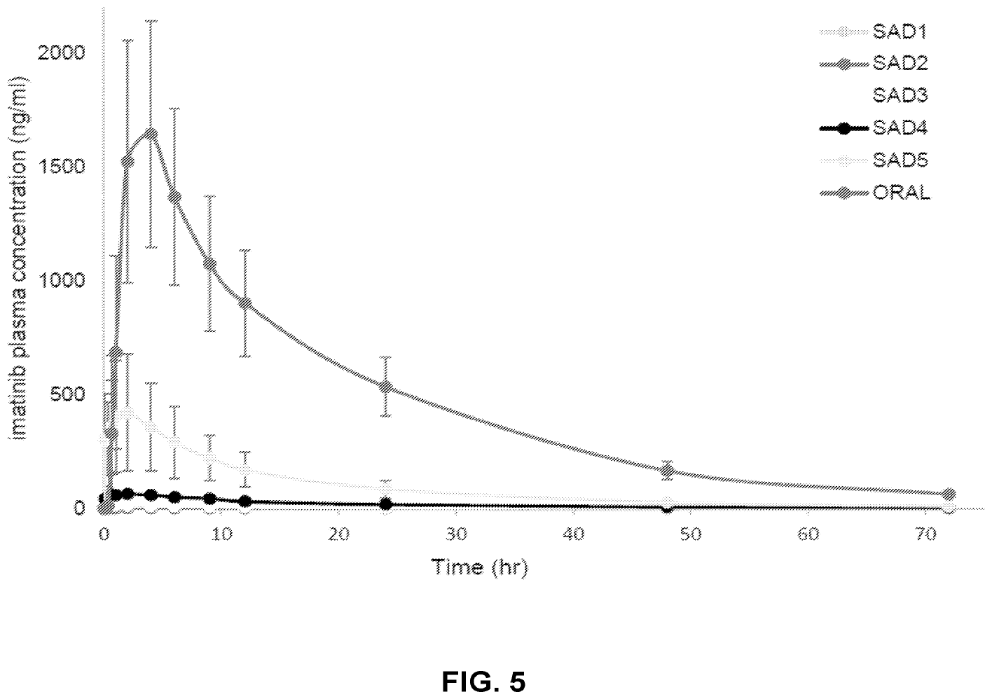

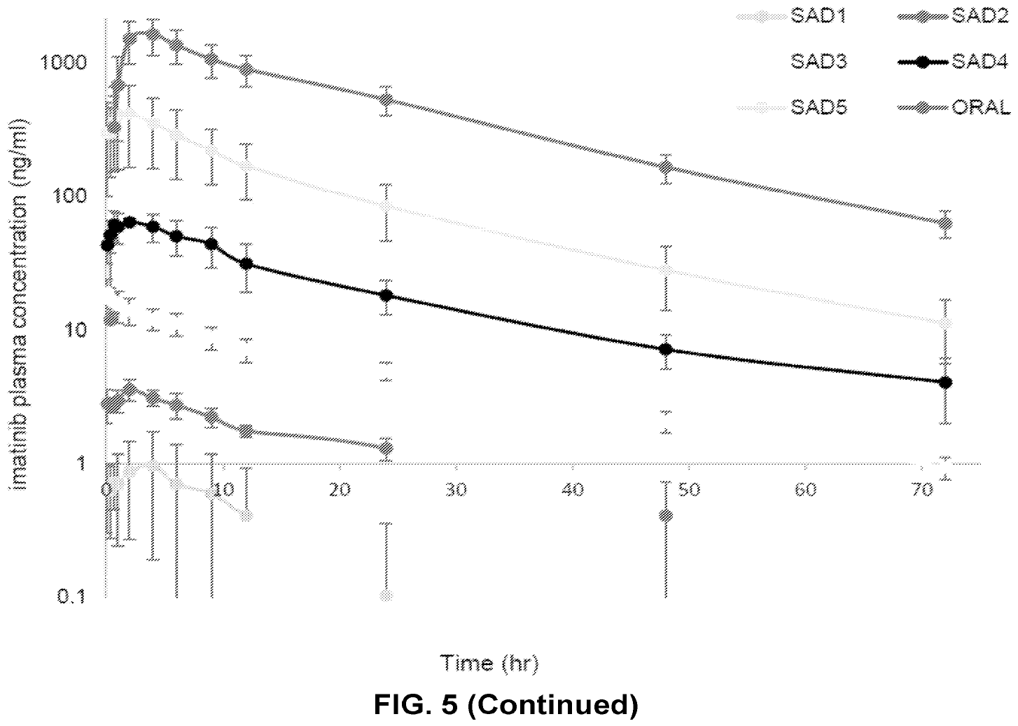

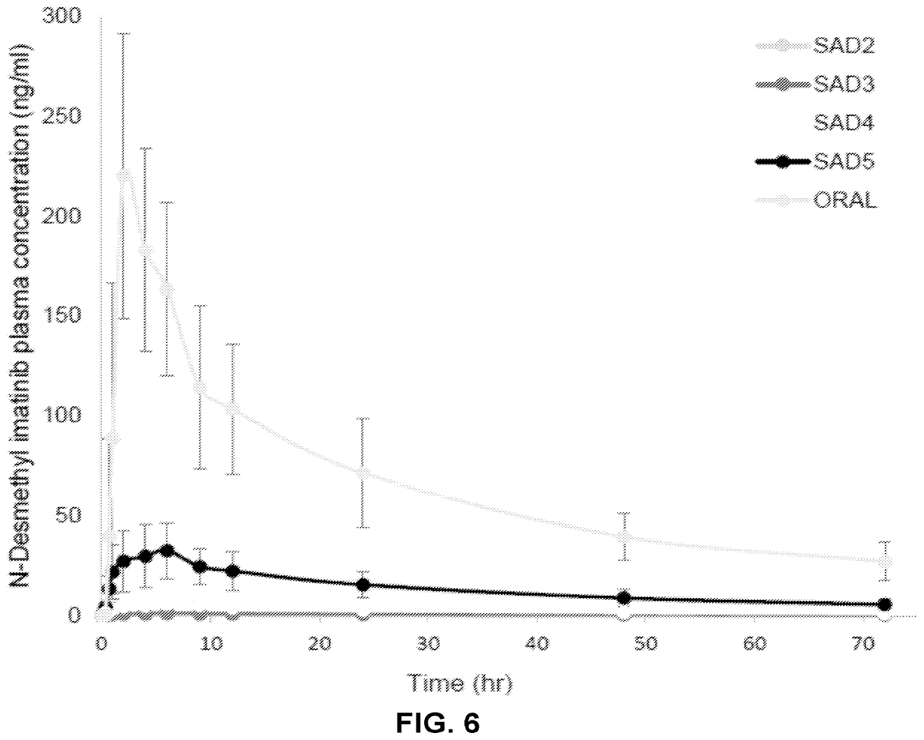

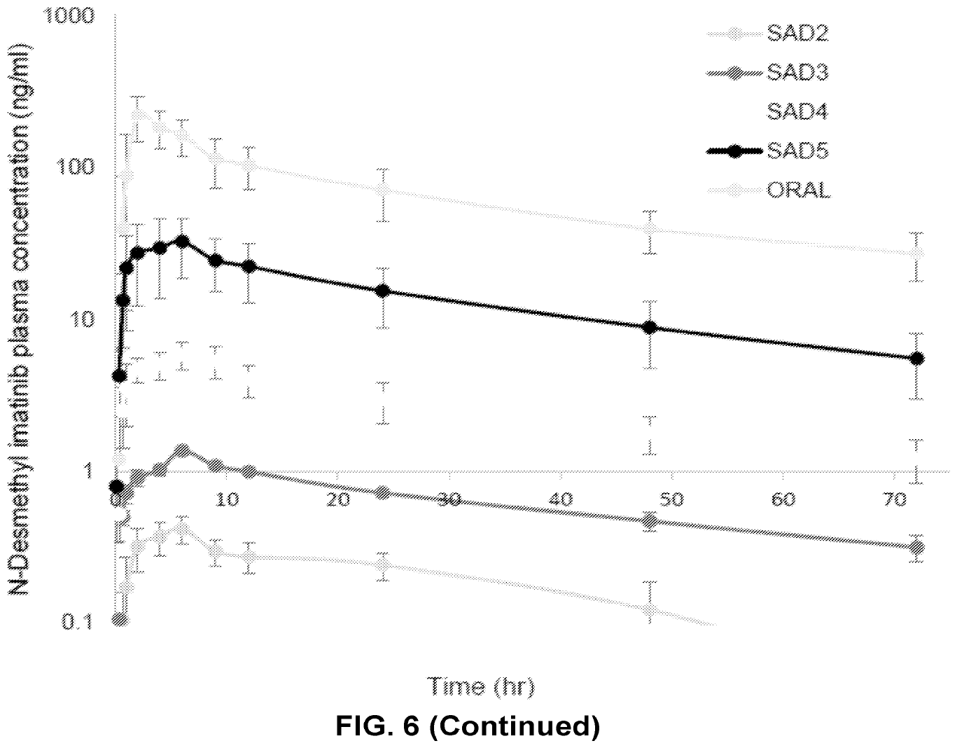

through 72 hours following dosing. Results are shown in Table 1 showing observed concentrations of imatinib and the imatinib metabolite, N-desmethyl imatinib. FIG. 5 illustrates imatinib plasma concentration over time for the various cohorts in the SAD study (standard and semi-logarithmic scales). FIG. 6 illustrates imatinib metabolite N-desmethyl imatinib plasma concentration over time for the various cohorts in the SAD study (standard and semi-logarithmic scales). SAD1 through SAD5 refer to the 1 mg through 90 mg cohorts respectively in FIGS. 5 and 6.

TABLE 1
Imatinib PK Parameters Following Single Administration
of Inhaled AV-101 or Oral Imatinib (Gleevec)
Imatinib

| | | | | | | Oral |
|---|---|---|---|---|---|---|
| | | | | | | Formulation |
| | AV-101 Inhalation (SAD) | | | | | (SAD) |
| | 1 mg | 3 mg | 10 mg | 30 mg | 90 mg | 400 mg |
| | AV-101 | AV-101 | AV-101 | AV-101 | AV-101 | Imatinib |
| Parameter | (N = 6)^{b} | (N = 6) | (N = 6) | (N = 6) | (N = 6) | (N = 8) |
| Cₘₐₓ | 1.161 | 3.805 | 20.198 | 73.771 | 423.831 | 1712.1 |
| (ng/mL) | (0.578) | (0.513) | (4.826) | (9.159) | (253.118) | (483.7) |
| Tₘₐₓ | 3.00 | 2.02 | 0.18 | 2.04 | 2.08 | 4.00 |
| (h)^{a} | (0.08-4.00) | (0.10-2.02) | (0.08-1.10) | (0.70-6.12) | (1.10-2.08) | (2.00-4.00) |
| AUC₀₋ₜ | 9.901 | 65.446 | 318.966 | 1279.330 | 6673.892 | 32665.8 |
| (ng*h/mL) | (10.925) | (13.672) | (54.025) | (306.032) | (3251.768) | (7641.0) |
| MRT₀₋ₜ | 4.79 | 14.99 | 20.97 | 18.38 | 17.18 | 19.54 |
| (h) | (3.14) | (4.19) | (0.70) | (2.12) | (2.31) | (1.20) |
| t_{1/2} | 9.00 | 19.60 | 19.95 | 19.27 | 16.05 | 15.43 |
| (h) | (2.20) | (4.02) | (1.06) | (2.89) | (2.47) | (1.37) |

N-Desmethyl Imatinib

| | | | | | | Oral |
|---|---|---|---|---|---|---|
| | | | | | | Formulation |
| | AV-101 Inhalation (SAD) | | | | | (SAD) |
| | 1 mg | 3 mg | 10 mg | 30 mg | 90 mg | 400 mg |
| | AV-101 | AV-101 | AV-101 | AV-101 | AV-101 | Imatinib |
| Parameter | (N = 4)^{c} | (N = 6) | (N = 6) | (N = 6) | (N = 6) | (N = 8) |
| Cₘₐₓ | 0.148 | 0.418 | 1.364 | 6.153 | 33.191 | 228.9 |
| (ng/mL) | (0.076) | (0.085) | (0.266) | (1.324) | (14.662) | (61.3) |
| Tₘₐₓ | 6.00 | 6.02 | 6.00 | 6.07 | 6.08 | 2.00 |
| (h)^{a} | (4.00-6.17) | (6.02-6.03) | (6.00-6.00) | (1.03-9.02) | (2.08-9.10) | (2.00-4.00) |
| AUC₀₋ₜ | 2.601 | 11.834 | 45.793 | 182.602 | 986.515 | 4783.9 |
| (ng*h/mL) | (NR) | (4.139) | (8.619) | (45.692) | (433.635) | (1457.3) |
| MRT₀₋ₜ | 8.97 | 21.96 | 28.40 | 25.25 | 26.36 | 24.89 |
| (h) | (NR) | (7.01) | (0.98) | (2.83) | (1.47) | (1.16) |
| t_{1/2} | NR | 34.75 | 40.54 | 32.24 | 31.86 | 32.53 |
| (h) | (NC) | (9.22) | (8.42) | (3.52) | (5.55) | (4.49) |

Abbreviations: Max = maximum; Min = minimum; N = number of observations; NC = not calculated; NR = not reportable; SAD = single ascending dose
^{a}Descriptive statistics are presented with mean ± SD. Tₘₐₓ is presented as Median (Min-Max)
^{b}n = 3 for t_{1/2};
^{c}n = 2 for AUC₀₋ₜ and MRT₀₋ₜ; and n = 1 for t_{1/2}

### Part 2: Multiple Ascending Dose (MAD) Study in Healthy Volunteers.

The MAD portion included three cohorts with 8-9 active participants in each cohort. AV-101 was be administered by inhalation BID for 7 days. Doses were determined by the results of the SAD portion as 10 mg, 30 mg, and 90 mg.

Blood samples for PK evaluation were taken prior to and through 12 hours following the first dose, prior to each dose on days 2-6, and prior to and through 12 hours following the last dose on Day 7. All subjects had blood taken for PK assessment at 24, 48, and 72 hours post the final dose on Day 7. Results are shown in Tables 2 (day 1) and 3 (day 7).

TABLE 2
Imatinib Pharmacokinetic Parameters Following Single Inhaled Administration
of AV-101 or Simulated 400 mg Oral Administration of Imatinib
Imatinib

| | AV-101 Inhalation (MAD, Day1) | | | | | | Oral Imatinib |
|---|---|---|---|---|---|---|---|
| | 10 mg AV-101 | | 30 mg AV-101 | | 90 mg AV-101 | | Simulation of |
| Parameter | (N = 8) | | (N = 9) | | (N = 9) | | 400 mg (Day 1) |
| Cₘₐₓ (ng/mL) | 24.149 | (10.205) | 88.689 | (31.273) | 262.364 | (81.875) | 1538 |
| Tₘₐₓ (h)^{a} | 0.48 | (0.08-2.00) | 0.10 | (0.10-2.02) | 4.08 | (0.37-6.08) | 3.4 |
| AUC₀₋₁₂ (ng*h/mL) | 181.783 | (90.368) | 672.909 | (291.868) | 2208.140 | (472.649) | 19432^{b} |
| N-Desmethyl Imatinib (MAD, Day1) | | | | | | | |
| | 10 mg AV-101 | | 30 mg AV-101 | | 90 mg AV-101 | | |
| Parameter | (N = 8) | | (N = 9) | | (N = 9) | | |
| Cₘₐₓ (ng/mL) | 2.014 | (1.115) | 6.877 | (2.885) | 26.005 | (11.372) | |
| Tₘₐₓ (h) | 6.00 | (2.00-6.00) | 6.02 | (2.02-6.02) | 6.08 | (4.08-6.08) | |
| AUC₀₋₁₂ (ng*h/mL) | 18.767 | (11.054) | 64.740 | (28.312) | 230.459 | (89.085) | |

Abbreviations: MAD = multiple ascending dose; Max = maximum; Min = minimum; N = number of observations
^{a}Descriptive statistics are presented with mean ± SD. Tₘₐₓ is presented as Median (Min-Max).
^{b}AUC₀₋₂₄

TABLE 3
Imatinib Pharmacokinetic Parameters Following Multiple Inhaled Administration
of AV-101 or Simulated 400 mg Oral Administration of Imatinib
Imatinib

| | AV-101 Inhalation (MAD, Day 7) | | | | | | Oral Imatinib |
|---|---|---|---|---|---|---|---|
| | 10 mg AV-101 | | 30 mg AV-101 | | 90 mg AV-101 | | Simulation of |
| Parameter | (N = 8) | | (N = 9) | | (N = 8) | | 400 mg (Day 7) |
| C_{max, ss} (ng/mL) | 69.171 | (31.052) | 214.770 | (88.164) | 890.363 | (236.758) | 2155 |
| Cₐᵥ (ng/mL) | 49.134 | (24.018) | 159.637 | (64.480) | 663.767 | (208.472) | 1251 |
| Tₘₐₓ (h)a | 0.67 | (0.08-2.00) | 0.10 | (0.10-2.02) | 1.58 | (0.17-2.08) | 3.3 |
| C_{min, ss} (ng/ml) | 31.741 | (14.312) | 118.214 | (47.427) | 533.634 | (183.392) | NAV |
| AUCₜₐᵤ (ng*h/mL) | 589.608 | (288.214) | 1915.638 | (773.754) | 7965.209 | (2501.658) | 30033^{b} |
| Fluctuation (%) | 78.28 | (11.09) | 70.30 | (8.78) | 67.18 | (16.18) | 115 |
| Rac(Cₘₐₓ) | 2.96 | (0.81) | 2.51 | (0.82) | 3.43 | (0.38) | NAV |
| Rac(AUC) | 3.34 | (0.65) | 2.98 | (0.69) | 3.53 | (0.48) | NAV |
| N-Desmethyl Imatinib (MAD, Day 7) | | | | | | | |
| | 10 mg AV-101 | | 30 mg AV-101 | | 90 mg AV-101 | | |
| Parameter | (N = 8) | | (N = 9) | | (N = 8) | | |
| C_{max, ss} (ng/ml) | 9.926 | (6.514) | 30.048 | (12.514) | 116.616 | (48.295) | |
| Tₘₐₓ (h)^{a} | 1.00 | (0.67-6.00) | 1.07 | (0.68-9.02) | 2.08 | (1.08-9.08) | |
| C_{min, ss} (ng/ml) | 6.626 | (3.932) | 22.613 | (8.969) | 83.469 | (38.761) | |
| AUCₜₐᵤ (ng*h/mL) | 100.217 | (64.338) | 322.167 | (134.665) | 1198.451 | (532.558) | |
| Rac(Cₘₐₓ) | 4.87 | (1.20) | 4.44 | (0.84) | 4.52 | (0.90) | |
| Rac(AUC) | 5.31 | (1.05) | 5.08 | (0.98) | 5.06 | (1.09) | |

Abbreviations: MAD = multiple ascending dose; Max = maximum; Min = minimum; N = number of observations; NAV = not available
^{a}Tₘₐₓ is presented as Median (Min-Max)

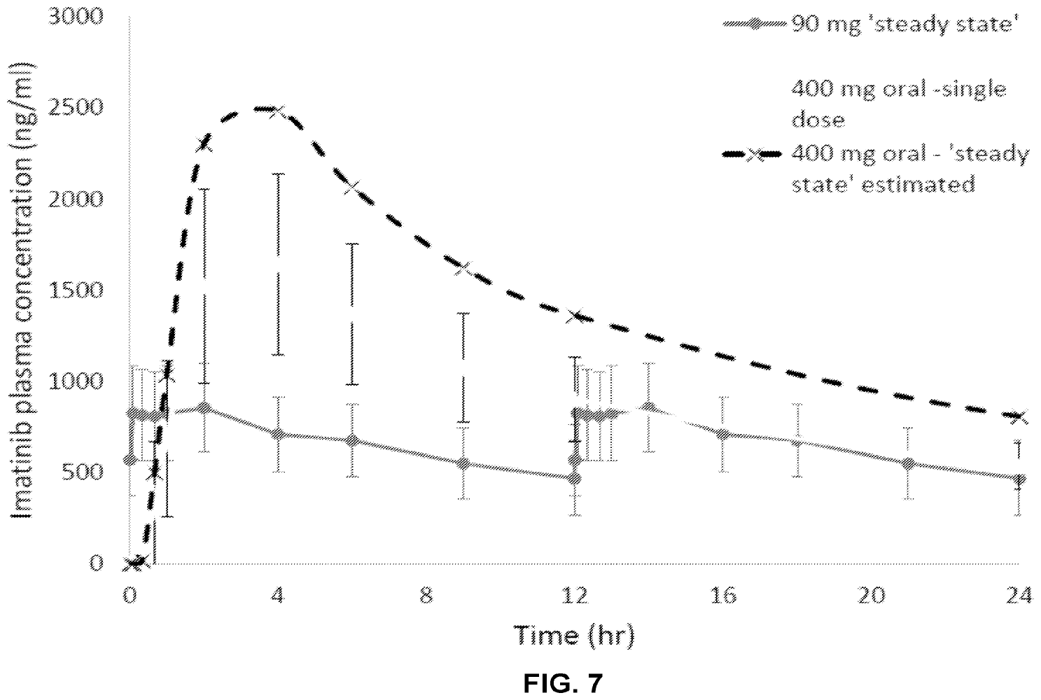

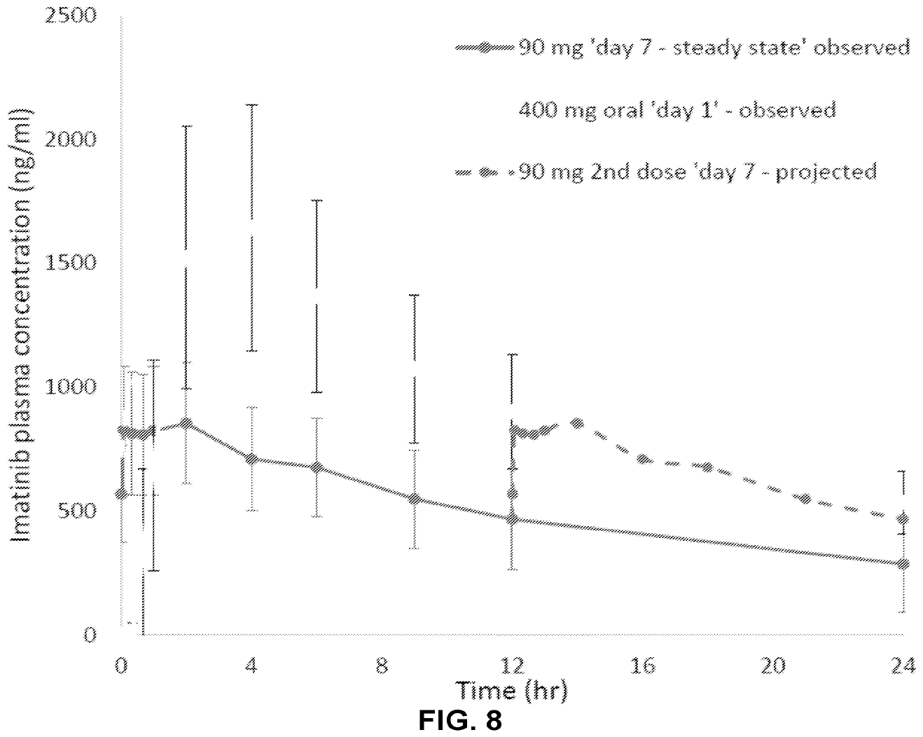

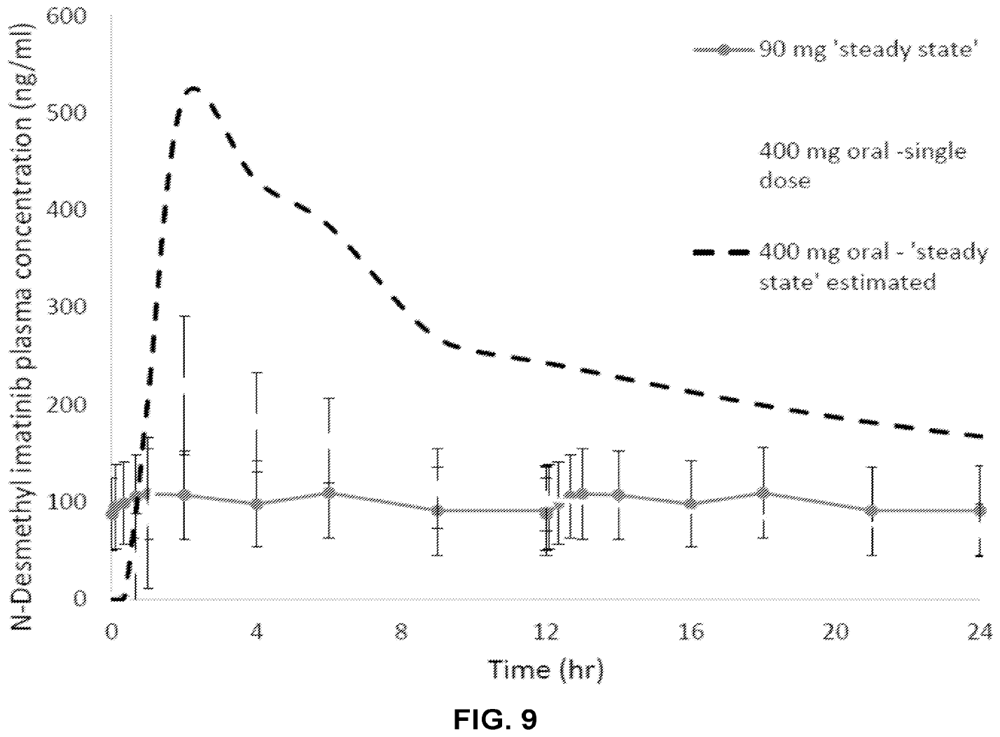

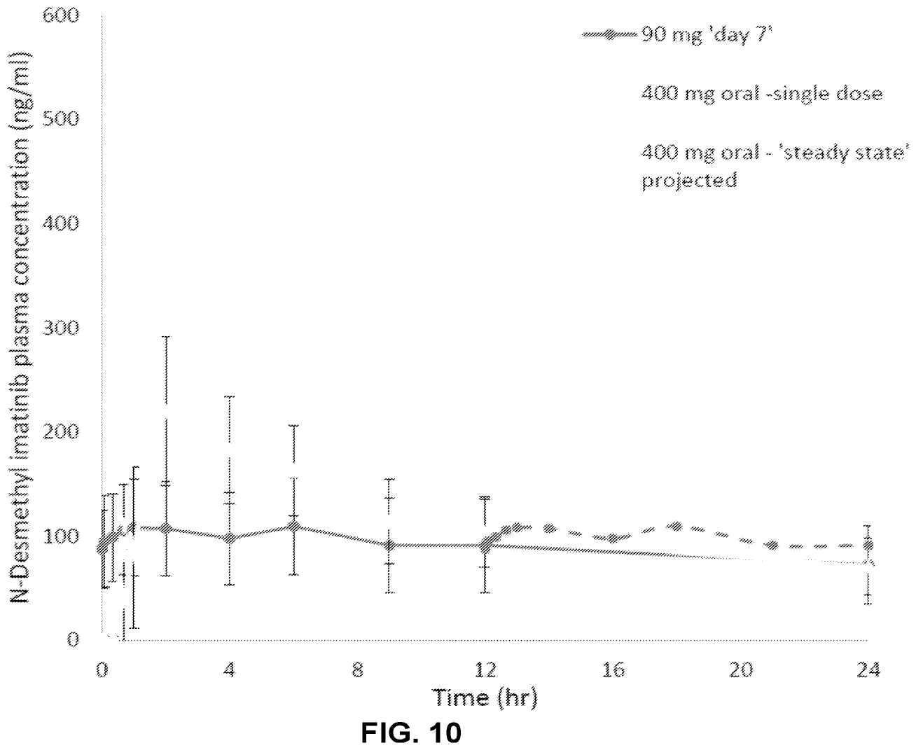

FIG. 7 shows imatinib plasma concentration of a 90 mg steady state dose from the MAD study compared to 400 mg oral single and steady state doses (estimated). FIG. 8 shows imatinib plasma concentration of a 90 mg steady state dose at day 7 of the multiple ascending dose study compared to 400 mg oral single dose observed on day 1 and concentration for a projected second dose at 90 mg on day 7. FIG. 9 shows N-desmethyl imatinib plasma concentration of a 90 mg steady state dose from the MAD study compared to 400 mg oral single and steady state doses (estimated). FIG. 10 shows N-desmethyl imatinib plasma concentration of a 90 mg dose at day 7 of the multiple ascending dose study compared to 400 mg oral single and steady state doses (projected).

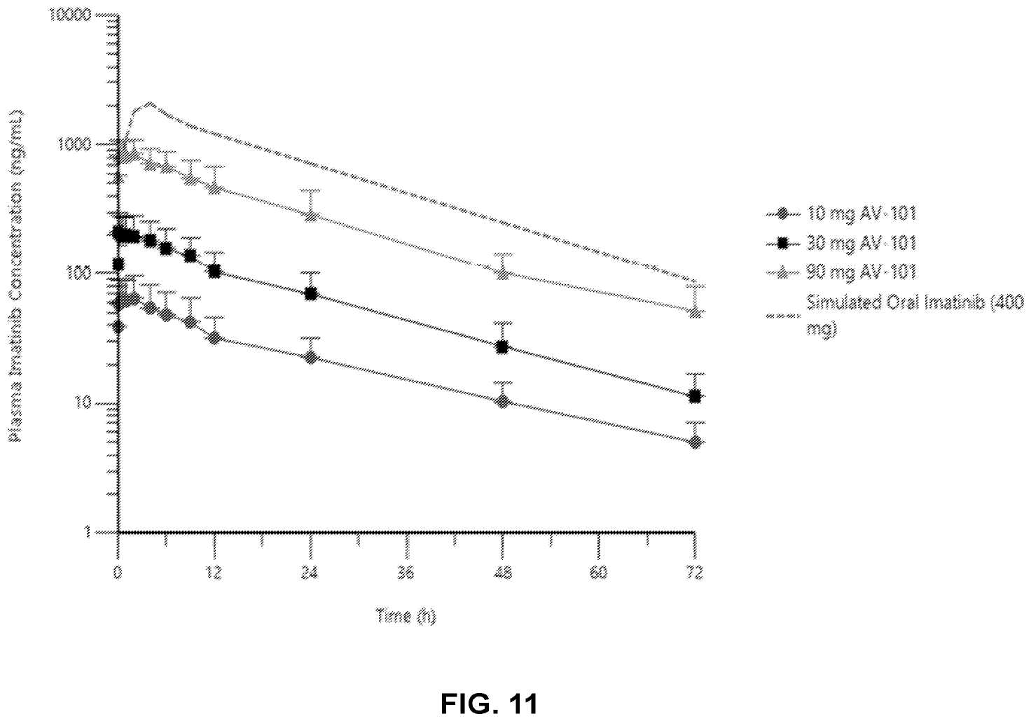

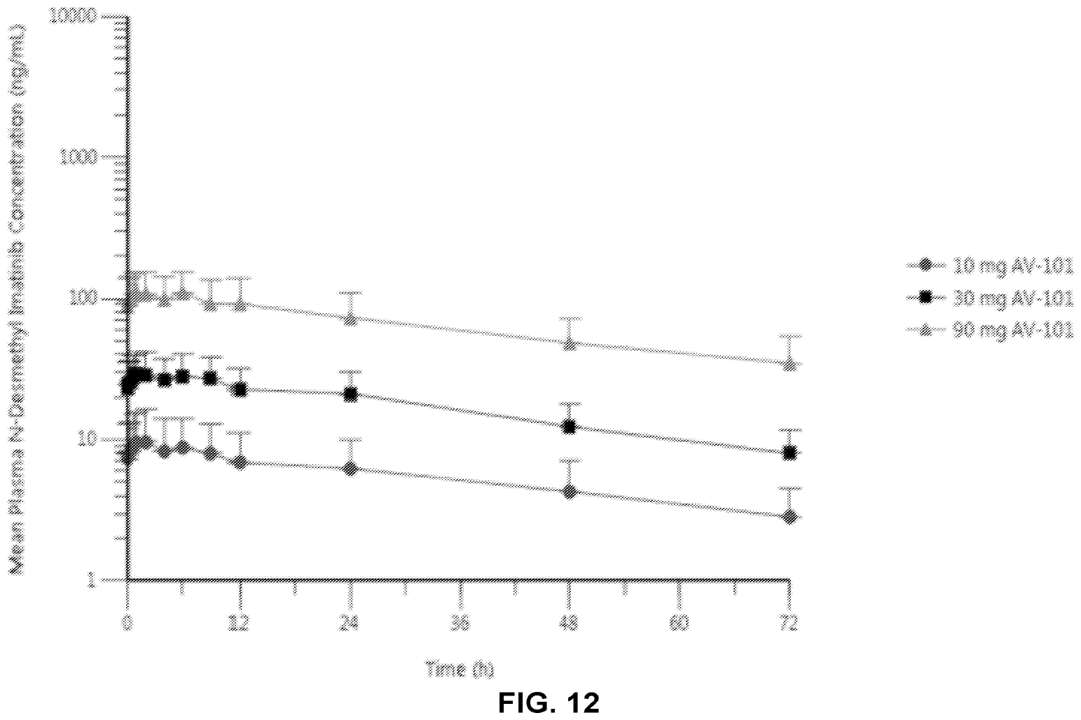

FIGS. 11 and 12 show concentration by time following multiple administration of inhaled AV-101 or simulated profile for 400 mg once daily imatinib with a semi-logarithmic scale for imatinib (FIG. 11) and N-desmethyl imatinib (FIG. 12). Concentrations are presented as mean ±SD over 72-hour on Day 7. Inhaled AV-101 concentrations by time were obtained during Day 7 of the MAD part. The simulated profile in FIGS. 11 and 12 shows concentration by time on day 7 following once daily oral dosing of 400 mg imatinib obtained from the population PK model developed from the observed data of the cohort that received Imatinib (Gleevec) oral during the SAD part.

### Example 2: Inhalable Imatinib Pharmacokinetic Testing

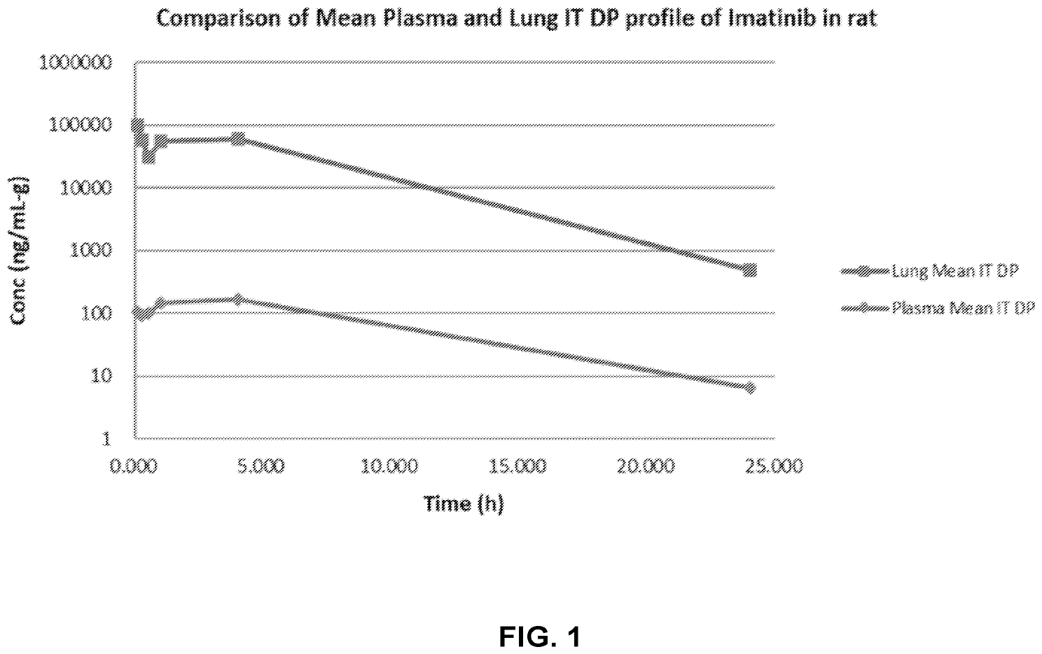

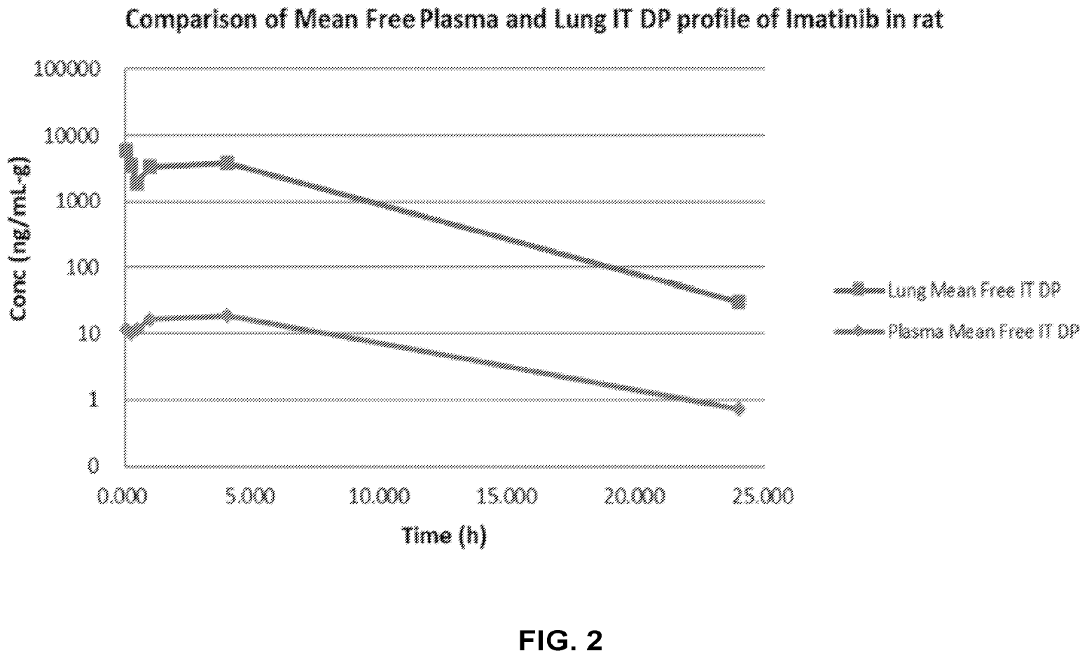

Imatinib was administered intratracheally to rats at 3 mg/kg in dry powder (IT DP) and suspension (IT) formats. Imatinib was administered orally as well for comparison. The rats were sacrificed at various time points after administration and plasma and lung concentrations were measured. FIG. 1 shows a comparison of mean imatinib concentrations in plasma and the lungs over time. FIG. 2 shows a comparison of mean free imatinib concentrations in plasma and the lungs over time. As shown, lung concentrations were much higher than plasma concentrations, showing the advantage of inhalation of imatinib for treatment of pulmonary disorders such as PAH while avoiding some of the adverse event risks associated with high systemic concentrations.

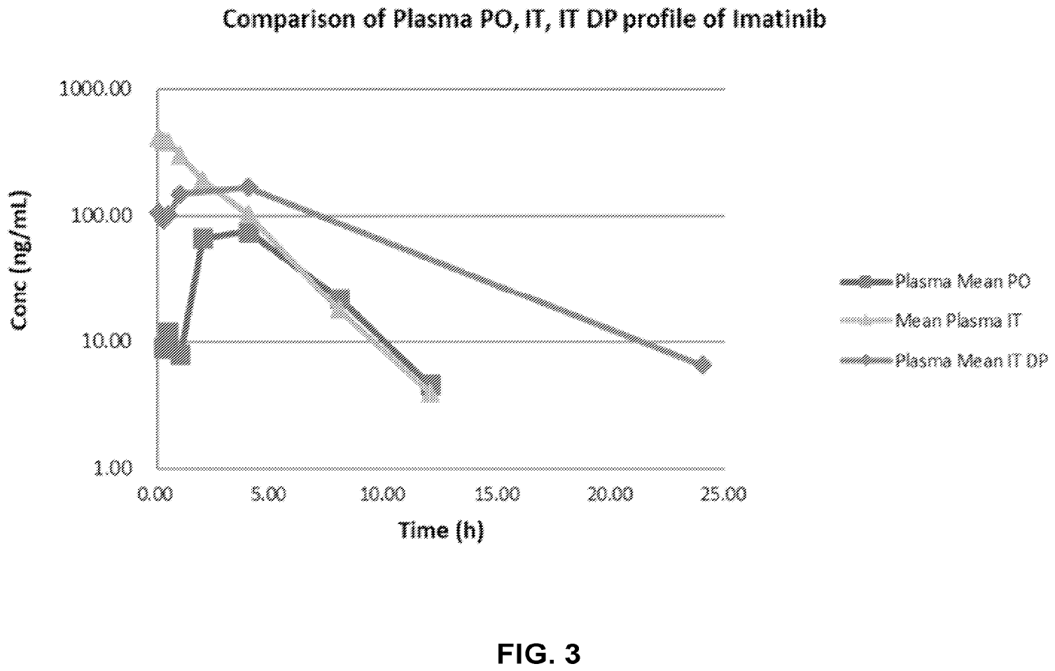

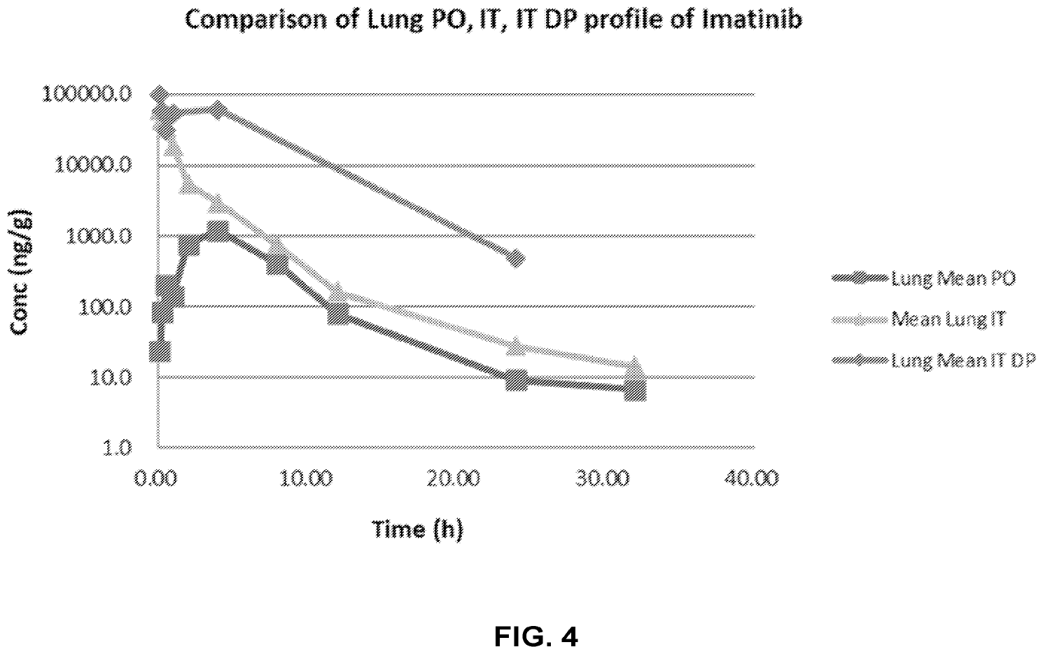

FIG. 3 shows a comparison of plasma concentration over time for intratracheally administered imatinib suspension (IT) and dry powder imatinib (IT DP) to orally administered imatinib (PO). FIG. 4 shows a comparison of lung concentrations for the same formulations. As shown, dry powder imatinib exhibits longer retention in the lungs. Compared to the orally administered imatinib, both intratracheal administrations result in a higher peak concentration in the lung tissue.

What is claimed is:

1. An inhalable formulation comprising at least 1 mg of imatinib.

2. The inhalable formulation of claim 1, wherein the imatinib is present in the formulation in an amount selected from the group consisting of 1 mg, 3 mg, 10 mg, 30 mg, 35 mg, 70 mg, and 90 mg.

3. The inhalable formulation of claim 1, wherein the inhalable formulation further comprises a carrier.

4. The inhalable formulation of claim 3, wherein the carrier is lactose.

5. The inhalable formulation of claim 1, wherein the inhalable formulation is a dry powder.

6. A combination product comprising:
a dry powder inhaler; and
a dry powder formulation comprising at least 1 mg of imatinib.

7. The combination product of claim 6, wherein the imatinib is present in the formulation in an amount selected from the group consisting of 1 mg, 3 mg, 10 mg, 30 mg, 35 mg, 70 mg, and 90 mg.

8. The combination product of claim 6, wherein the dry powder formulation further comprises a carrier.

9. The combination product of claim 8, wherein the carrier is lactose.

10. The combination product of claim 6, wherein the dry powder formulation is packaged in a capsule sized and configured to be inserted into the dry powder inhaler.

11. A method of treating a condition of the pulmonary cardiovascular system, the method comprising providing a pharmaceutical composition to a human subject having a condition of the pulmonary cardiovascular system, wherein the pharmaceutical composition is an inhalable formulation that comprises at least 1 mg of imatinib.

12. The method of claim 11, wherein the condition of the pulmonary cardiovascular system is pulmonary arterial hypertension (PAH).

13. The method of claim 11, wherein the pharmaceutical composition is provided once daily.

14. The method of claim 11, wherein the pharmaceutical composition is provided twice daily.

15. The method of claim 11, wherein the pharmaceutical composition comprises 1 mg of imatinib.

16. The method of claim 11, wherein the pharmaceutical composition comprises 3 mg of imatinib.

17. The method of claim 11, wherein the pharmaceutical composition comprises 10 mg of imatinib.

18. The method of claim 11, wherein the pharmaceutical composition comprises 30 mg of imatinib.

19. The method of claim 11, wherein the pharmaceutical composition comprises 35 mg of imatinib.

20. The method of claim 11, wherein the pharmaceutical composition comprises 70 mg of imatinib.

21. The method of claim 11, wherein the pharmaceutical composition comprises 90 mg of imatinib.

22. The method of claim 11, wherein the inhalable formulation further comprises a carrier.

23. The method of claim 22, wherein the carrier is lactose.

24. The method of claim 11, wherein the inhalable formulation is a dry powder.

25. The method of claim 24, providing the pharmaceutical composition as a dry powder inhaler loaded with the dry powder formulation of imatinib.
